(19) United States
(12) Patent Application Publication
(10) Pub. No.: US 2023/0210403 A1
(43) Pub. Date: Jul. 6, 2023

(54) A MECHANOMYOGRAPHY APPARATUS AND ASSOCIATED METHODS

(71) Applicant: IMPERIAL COLLEGE INNOVATIONS LIMITED, London (GB)

(72) Inventors: Ravi Vaidyanathan, London (GB); Farokh S. Atashzar, London (GB); Sebastian Carlos Mancero-Castillo, London (GB); Samuel Wilson, London (GB)

(21) Appl. No.: 18/012,468

(22) PCT Filed: Jul. 2, 2021
(86) PCT No.: PCT/GB2021/051690

§ 371 Date: Dec. 22, 2022

(30) Foreign Application Priority Data
Jul. 3, 2020 (GB) 2010270.3

(51) Int. Cl. A61B 5/11; A61B 5/00; A61B 5/296; A61B 5/389

(52) U.S. Cl. CPC A61B 5/1107; A61B 5/6843; A61B 5/296; A61B 5/389; A61B 5/7289; A61B 2562/0247; A61B 2562/046; A61B 2562/066; A61B 2562/164; A61B 2562/0204

(57) ABSTRACT

An apparatus configured for application to a surface of a body, the apparatus comprising:
an array of mechanomyography sensors spatially distributed across a substrate, each mechanomyography sensor configured to detect mechanomyography signals from the body to which the apparatus is applied; and a pressure bias system configured to provide a variation in contact pressure of the mechanomyography sensors to the body surface to receive mechanomyography signals at different levels of applied contact pressure.

### TECHNICAL FIELD

The present disclosure relates to the monitoring of muscle activity in human or animal bodies and, in particular, concerns an apparatus and associated methods for monitoring said muscle activity.

### BACKGROUND

Surface electromyography (sEMG) has been used for decades as the standard myographic modality for recording and monitoring muscle activity. However, there are still challenges with the use of this modality, such as variations due to electrode-skin impedance, sensor positioning, the need for direct contact with the skin, and expensive hardware requirements for electromagnetic noise cancelation and sophisticated signal amplification.

As an alternative, mechanomyography (MMG) has attracted a great deal of interest over the past years as it is a low-cost myographic modality which records the mechanical responses of the muscle activation during contractions as opposed to the electrical activity of the motoneurons. These mechanical responses are captured in the form of oscillations produced by displacement and dimensional changes of muscle fibres that occur during a contraction. Using the MMG modality, some of the major difficulties related to the use of EMG can be addressed. This makes MMG an ideal modality for practical applications that require in-home compatibility, and a low-cost solution for muscle monitoring while being robust to electrode-skin conditions and requiring simple calibration and placement procedure with no need for skin preparation.

MMG has been studied in the literature in a single-modality format using different sensors such as accelerometers, piezoelectric contact sensors, and microelectromechanical (MEM) microphones, each of which has advantages and limitations. For example, accelerometers have been found to provide a better mechanical parameter for measuring vibrations compared to piezoelectric contact sensors thanks to the fact that the MMG signal acquired from the accelerometer provides a flatter spectrum. Similarly, microphone MMG has shown to be more robust to motion artefacts compared to accelerometers, thus making it ideal for dynamic tasks. However, there are different factors that affect the quality of the MMG modalities such as the thickness of the fat layers of the dermis. In addition, MMG has shown some difficulties for the assessment of motor unit activations when it is used as a stand-alone component since reading the vibration depends critically on the relative spatial location of motor units.

The listing or discussion of a prior-published document or any background in this specification should not necessarily be taken as an acknowledgement that the document or background is part of the state of the art or is common general knowledge. One or more aspects/embodiments of the present disclosure may or may not address one or more of the background issues.

### SUMMARY

According to a first aspect, there is provided an apparatus configured for application to a surface of a body, the apparatus comprising:
an array of mechanomyography sensors spatially distributed across a substrate, each mechanomyography sensor configured to detect mechanomyography signals from the body to which the apparatus is applied; and
a pressure bias system configured to provide a variation in contact pressure of the mechanomyography sensors to the body surface to receive mechanomyography signals at different levels of applied contact pressure.

The pressure bias system may be configured to spatially modulate the applied pressure across the sensor array.

The spatial modulation of applied pressure may be effected by a plurality of the mechanomyography sensors in the sensor array being distributed at different distances from and orthogonal to a reference plane of the substrate.

The pressure bias system may comprise a plurality of platforms on the substrate at different heights relative to the reference plane of the substrate. Each platform may bear at least one mechanomyography sensor of the sensor array.

The pressure bias system may be configured to temporally modulate the applied pressure of at least some of the mechanomyography sensors in the sensor array.

The temporal modulation of applied pressure may be effected by at least some of the mechanomyography sensors having an adjustable height relative to a reference plane of the substrate.

The pressure bias system may comprise one or more actuators or inflatable elements configured for adjusting the height of at least one mechanomyography sensor relative to the reference plane of the substrate.

The substrate may be configured to be worn around the body, and the pressure bias system may comprise means for adjusting the tightness of the substrate around the body.

The means for adjusting the tightness of the substrate around the body may comprise one or more of a fastener and an inflatable chamber attached to the substrate.

The substrate may comprise a plurality of rigid substrate portions linked together by flexible and/or stretchable connectors.

The array of mechanomyography sensors may comprise one or more of acoustic sensors, accelerometers, piezoelectric sensors and force sensors. The accelerometers may be one-dimensional, two-dimensional or three-dimensional accelerometers.

The apparatus may further comprise an array of pressure sensors. Each pressure sensor may be configured to provide an indication of the contact pressure of a respective mechanomyography sensor as applied to the body surface.

The array of pressure sensors may comprise one or more of a piezoresistive pressure sensor, a strain gauge pressure sensor, a capacitive pressure sensor, a potentiometric pressure sensor, an inductive pressure sensor, a resonant pressure sensor, an electromagnetic pressure sensor, a variable reluctance pressure sensor, and an optical pressure sensor.

The apparatus may further comprise an array of electromyography electrodes disposed on the substrate. Each electromyography electrode may be configured to detect electromyography signals from the body to which the apparatus is applied.

The array may be a one-dimensional or two-dimensional array.

The array may be a regular or irregular array.

The mechanomyography sensors of the array may have a spacing of no more than 5 mm, 10 mm, 15 mm or 20 mm. The same spacing may apply to the electromyography sensors and/or pressure sensors.

The body may comprise at least part of a human or animal body.

The apparatus may comprise processing circuitry configured to process the mechanomyography signals detected from the body at different levels of applied contact pressure to determine activity associated with one or more muscles located within the body.

The determined activity associated with the one or more muscles within the body may comprise at least one of muscle activity, neural activity, neuronal activity and cerebral activity.

The determined muscle activity may comprise one or more biomechanical properties of the muscles.

The processing circuitry may be configured to process the detected mechanomyography signals to determine activity associated with one or more muscles located at different corresponding depths within the body.

The processing circuitry may be configured to process the detected mechanomyography signals to detect the frequency response of one or more muscles at different levels of applied contact pressure.

The processing circuitry may be configured to associate mechanomyography signals received from the mechanomyography sensors with pressure signals received from the respective pressure sensors.

The processing circuitry may be configured to associate the mechanomyography signals with the pressure signals by synchronising read-out of the mechanomyography sensors with read-out of the respective pressure sensors.

The processing circuitry may be configured to fuse the mechanomyography signals with the pressure signals.

The processing circuitry may comprise one or more processors or application-specific integrated circuits.

According to a second aspect, there is provided a method comprising:
applying the apparatus of the first aspect to a surface of a body;
detecting mechanomyography signals from the body at different levels of applied contact pressure; and
processing the detected mechanomyography signals to determine activity associated with one or more muscles located within the body.

The detected mechanomyography signals may be processed to determine a gesture, or an intent to make a gesture, of the body.

According to a third aspect, there is provided an apparatus configured for application to a surface of a body, the apparatus comprising:
a mechanomyography sensor configured to detect mechanomyography signals from the body to which the apparatus is applied;
a pressure sensor configured to provide an indication of contact pressure of the mechanomyography sensor on the body surface; and
processing circuitry configured to associate the detected mechanomyography signals with the indicated contact pressure for use in determining activity associated with a muscle located within the body.

The apparatus may further comprise a pressure bias system configured to provide an adjustable contact pressure of the mechanomyography sensor to the body surface to receive mechanomyography signals at different levels of applied contact pressure.

According to a fourth aspect, there is provided a method comprising:
applying the apparatus of the third aspect to a surface of a body;
detecting mechanomyography signals from the body at an indicated level of applied contact pressure; and
processing the detected mechanomyography signals to determine activity associated with one or more muscles located within the body.

According to a fifth aspect, there is provided apparatus configured for application to a surface of a body, the apparatus comprising:
an array of mechanomyography sensors spatially distributed across a substrate, each mechanomyography sensor configured to detect mechanomyography signals from the body to which the apparatus is applied,
wherein the mechanomyography sensors of the array have a spacing of no more than 20 mm.

The apparatus may further comprise a pressure bias system configured to provide a variation in contact pressure of the mechanomyography sensors to the body surface to receive mechanomyography signals at different levels of applied contact pressure.

According to a sixth aspect, there is provided a method comprising:
applying the apparatus of the fifth aspect to a surface of a body;
detecting mechanomyography signals from a single muscle or muscle group within the body using the array of mechanomyography sensors; and
processing the detected mechanomyography signals to determine activity associated with the muscle or muscle group.

According to a seventh aspect, there is provided an apparatus configured for application to a surface of a body, the apparatus comprising:
a mechanomyography sensor configured to detect mechanomyography signals from the body to which the apparatus is applied;
a pressure sensor integrated with the mechanomyography sensor and configured to provide an indication of contact pressure of the mechanomyography sensor on the body surface; and
processing circuitry configured to calibrate the detected mechanomyography signals using the indicated contact pressure for use in determining activity associated with a muscle located within the body.

The apparatus may comprise a casing configured to house the mechanomyography sensor, and an attachment member for attaching the casing to the body, and the pressure sensor may be positioned between the casing and the attachment member such that the contact pressure causes compression of the pressure sensor.

According to an eighth aspect, there is provided a method comprising:
applying the apparatus of the seventh aspect to a surface of a body;
calibrating mechanomyography signals detected from the body at an indicated level of applied contact pressure; and
processing the calibrated mechanomyography signals to determine activity associated with one or more muscles located within the body.

According to a ninth aspect, there is provided apparatus configured for application to a surface of a body, the apparatus comprising:
an array of mechanomyography sensors spatially distributed across a substrate with a spacing of no more than 20 mm, each mechanomyography sensor configured to detect mechanomyography signals from the body to which the apparatus is applied: and
processing circuitry configured to decompose the mechanomyography signals detected by the array of mechanomyography sensors for use in determining neural activity associated with one or more muscles located within the body.

According to a tenth aspect, there is provided a method comprising:
applying the apparatus of the ninth aspect to a surface of a body;
detecting mechanomyography signals from a single muscle or muscle group within the body using the array of mechanomyography sensors; and
decomposing the detected mechanomyography signals to determine neural activity associated with the muscle or muscle group.

According to an eleventh aspect, there is provided an apparatus as substantially described herein with reference to, and as illustrated by, the accompanying drawings.

The optional features described in relation to the apparatus of the first aspect are also applicable to the apparatus of the third, fifth, seventh, ninth and eleventh aspects where compatible.

The steps of any method disclosed herein do not have to be performed in the exact order disclosed, unless explicitly stated or understood by the skilled person.

Corresponding computer programs (which may or may not be recorded on a carrier) for implementing one or more of the methods disclosed herein are also within the present disclosure and encompassed by one or more of the described example embodiments.

The present disclosure includes one or more corresponding aspects, example embodiments or features in isolation or in various combinations whether or not specifically stated (including claimed) in that combination or in isolation. Corresponding means for performing one or more of the discussed functions are also within the present disclosure.

The above summary is intended to be merely exemplary and non-limiting.

### BRIEF DESCRIPTION OF THE FIGURES

A description is now given, by way of example only, with reference to the accompanying schematic drawings, in which:—

FIG. 1a shows an MMG apparatus (perspective view);

FIG. 1b shows the MMG apparatus of FIG. 1 further comprising a protective housing (perspective view);

FIG. 1c shows another MMG apparatus (perspective view);

FIG. 2a shows an MMG apparatus with a spatial modulation pressure bias system (cross-section);

FIG. 2b shows a perspective view of the MMG apparatus of FIG. 2a;

FIG. 3a shows an MMG sensor (cross-section);

FIG. 3b shows an MMG sensor with an integrated pressure sensor (cross-section);

FIG. 4a shows the MMG sensor of FIG. 3b with an integrated temporal modulation pressure bias system in a first state (cross-section);

FIG. 4b shows the MMG sensor of FIG. 4a with the temporal modulation pressure bias system in a second state (cross-section);

FIG. 5a shows the MMG sensor of FIG. 3b with another integrated temporal modulation pressure bias system in a first state (cross-section);

FIG. 5b shows the MMG sensor of FIG. 5a with the temporal modulation pressure bias system in a second state (cross-section);

FIG. 6a shows the MMG sensor of FIG. 3b with another integrated temporal modulation pressure bias system (perspective view);

FIG. 6b shows the MMG sensor of FIG. 6a in cross-section;

FIG. 7a shows another example of an MMG apparatus configured to be worn around the body;

FIG. 7b shows the MMG apparatus of claim 7a after the tightness has been adjusted;

FIG. 8 shows the temporal modulation pressure bias system of FIG. 6a configured to apply three different levels of contact pressure;

FIG. 9a shows the MMG apparatus of FIG. 7a applied to an able-bodied participant;

FIG. 9b shows the MMG apparatus of FIG. 7a applied to an amputee participant;

FIGS. 10a-g show the different hand gestures used for testing the MMG apparatus of FIG. 7a;

FIG. 11 shows the MMG signals detected during the experiment;

FIG. 12 shows the average confusion matrices for each level of contact pressure applied during the experiment;

FIG. 13a shows the percentage accuracy of the classifier vs level of contact pressure for able-bodied participants; and

FIG. 13b shows the percentage accuracy of the classifier vs level of contact pressure for amputee participants.

### DESCRIPTION OF SPECIFIC ASPECTS/EMBODIMENTS

As mentioned in the background section, there are a number of challenges with existing sEMG and MMG systems. There will now be described an apparatus and associated methods that may address one or more of these challenges. Other examples depicted in the figures have been provided with reference numerals that correspond to similar features of earlier described examples. For example, feature number 1 can also correspond to numbers 101, 201, 301 etc. These numbered features may appear in the figures but may not have been directly referred to within the description of these particular examples. These have still been provided in the figures to aid understanding of the further examples, particularly in relation to the features of similar earlier described examples.

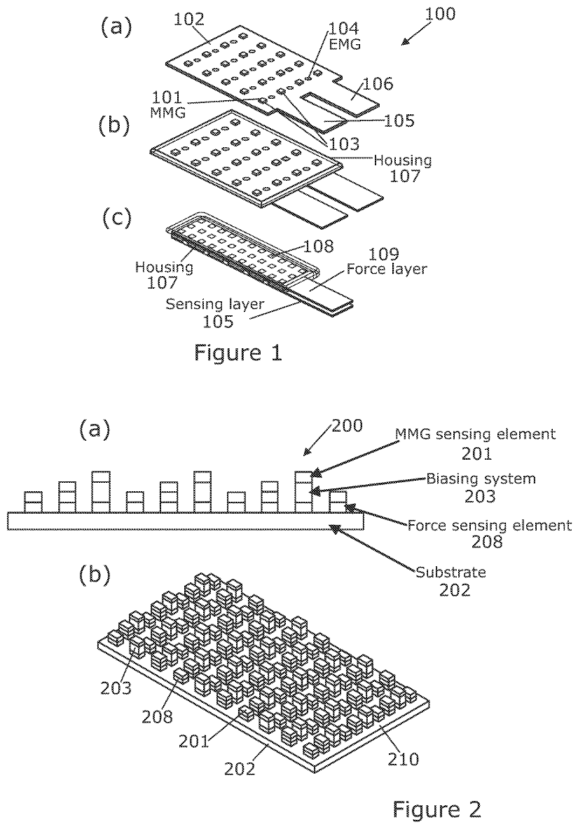

FIGS. 1a and 1b show one example of an apparatus 100 for application to a surface of a body. The term “body” as used herein may be taken to mean at least part of a human or animal body. The apparatus comprises an array of MMG sensors 101 spatially distributed across a substrate 102, each MMG sensor 101 configured to detect MMG signals from the body to which the apparatus 100 is applied. Although the MMG sensors 101 in this example form a regular array in two-dimensions, they could be distributed across the substrate 102 to form a regular or irregular array in one or two dimensions. As described in more detail later, the apparatus 100 further comprises a pressure bias system 103 configured to provide a variation in contact pressure of the MMG sensors 101 to the body surface to receive MMG signals at different levels of applied contact pressure. The term “contact pressure” as used herein may be taken to mean the static pressure (or force) applied by the pressure bias system 103 to the surface of the body during detection of the MMG signals.

It has been found that MMG sensors 101 have some degree of sensitivity to the contact pressure whereby an increase in the contact pressure at the point of contact with the skin can increase the amplitude and signal-to-noise ratio of the MMG signal. The contact pressure not only reduces the volume conduction effect (signal damping due to the tissue between the muscle and the sensor 101), but also results in a higher density of tissue (due to the non-linear viscoelastic characteristics of tissue) which increases the conductivity, and varies the frequency, of mechanical waves from the muscle to the skin.

The combination of the MMG sensor array 101 and the pressure bias system 103 of the present apparatus 100 therefore enables MMG signals to be detected from muscles located at different regions and depths beneath the surface of the body. This increases the spatial information context of the signal space relative to conventional MMG systems. It also allows the propagation of mechanical vibrations on the skin to be monitored.

As shown in FIGS. 1a and 1b, the apparatus 100 may also comprise an array of EMG electrodes 104 disposed on the substrate 102, each configured to detect EMG signals from the body to which the apparatus 100 is applied. This results in a hybrid system that can evaluate the electromechanical characteristics of motor unit activations. As with the MMG sensors 101, the EMG sensors 104 may be distributed regularly or irregularly across the substrate 102 to form a one-dimensional or two-dimensional array. The illustrated apparatus 100 also comprises separate electrical connectors 105, 106 for reading out the MMG 101 and EMG 104 sensors (e.g. for use by processing circuitry), and a protective housing 107 which prevents exposure of the sensors 101, 104 to the surrounding environment (shown in FIG. 1b).

FIG. 1c shows another example of the present apparatus 100. In addition to the MMG sensor array 101 (with or without the EMG sensors 104), the apparatus 100 further comprises an array of pressure/force sensors 108 each configured to provide an indication of the (local) contact pressure of a respective mechanomyography sensor 101 as applied to the body surface. Separate electrical connectors 105, 109 for reading out the MMG 101 and pressure 108 sensors are also illustrated. In this way, the processing circuitry may be configured to associate (e.g. synchronise based on timing information) the detected MMG signals with the indicated contact pressure for use in determining a physical property of a muscle located at a corresponding depth within the body. This association between the MMG signals and the corresponding contact pressure at which they were obtained is important for consistency between repeated tests. This is especially critical for detecting gesture (or gesture intent, for amputees) where inconsistencies between the test and training data sets can result in incorrect classification. The indicated contact pressure may also be used to calibrate the associated MMG signals.

The MMG sensors 101 may have a spacing of no more than 5 mm, 10 mm, 15 mm or 20 mm. The proximity of adjacent sensors enables multiple sensors to detect MMG signals from the same muscle or muscle group within the body, thereby increasing the resolution of the system. This sensor spacing also means that the apparatus 100 is more robust if some of the MMG sensors 101 in the array are defective. The same spacing may also be used for the EMG 104 and/or pressure 108 sensor arrays.

FIGS. 2a and 2b show an example of the present apparatus 200 in which the pressure bias system 203 is configured to spatially modulate the applied pressure across the MMG sensor array 201. The spatial modulation of applied pressure may be effected by a plurality of the MMG sensors 201 in the sensor array being distributed at different distances from and orthogonal to a reference plane of the substrate 202. To achieve this, the pressure bias system 203 of the illustrated example comprises a plurality of platforms on the substrate 202 at different heights relative to the reference plane (e.g. the upper surface 210) of the substrate 202, each platform bearing a respective MMG sensor 201 of the sensor array. In another example, each platform could be configured to support more than one MMG sensor 201. Furthermore, the substrate 202 itself could be rigid or flexible. As such, the upper surface 210 may be planar or curved.

The apparatus 200 of FIGS. 2a and 2b also comprises an array of pressure sensors 208 as described above. Although the platforms of the pressure bias system 203 are shown positioned between the respective MMG 201 and pressure sensors 208, different stacking orders could be used instead (e.g. by switching the order of the platforms 203 and pressure sensors 208).

The array of MMG sensors 201 described herein may comprise one or more of acoustic sensors, accelerometers, piezoelectric sensors and force sensors. The accelerometers may be configured to measure in one, two or three dimensions. The primary component of the MMG signal is perpendicular to the skin, but components such as the large muscle movements and others may have non-perpendicular components which can be observed using multi-dimensional MMG sensors. The force sensors are configured to detect pressure changes upon muscle activation due to changes in muscle volume and are not to be confused with the pressure sensors 208 described herein for measuring the static pressure applied by the pressure bias system 203 to the body surface.

The array of pressure sensors 208 may comprise one or more of piezoresistive pressure sensors, strain gauge pressure sensors, capacitive pressure sensors, potentiometric pressure sensors, inductive pressure sensors, resonant pressure sensors, electromagnetic pressure sensors, variable reluctance pressure sensors, and optical pressure sensors. The use of multiple types of MMG 201 and/or pressure sensor 208 within the same apparatus 200 could help to address the limitations associated with any one modality.

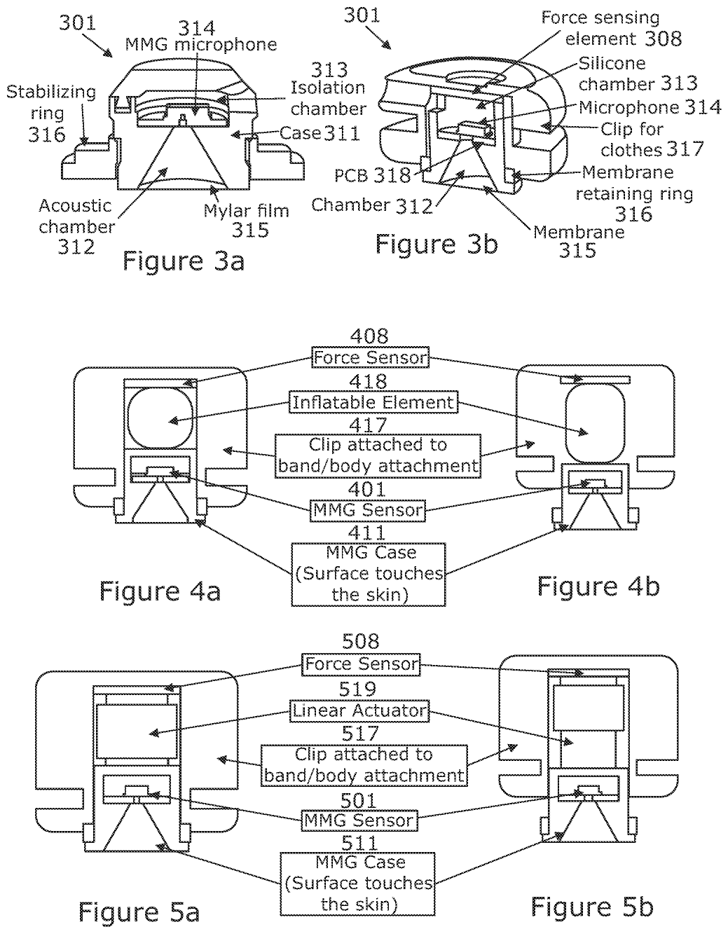

FIG. 3a shows one example of an acoustic MMG sensor 301. The sensor 301 consists of four components: a case/casing 311 used to hold all of the parts together and defining an acoustic chamber 312 and an isolation chamber 313, a microphone 314 used to capture the MMG activity, a portion of transparent mylar film 315 used to amplify the changes in pressure within the acoustic chamber 312, and a stabilizing ring 316 which dimensions have been adjusted to snap-fit around the frontal side of the case 311. The stabilizing ring 316 causes the mylar film 315 to remain firmly stretched at the same time as preventing the case 311 from shifting or tilting. As the mylar film 315 is excited by propagated muscle vibration, changes in air pressure within the acoustic chamber 312 are captured by the microphone 314. The microphone 314 itself is positioned on the backside of the case 311 at the bottom of the isolation chamber 313 which is sealed and filled with glue.

FIG. 3b shows another example of an acoustic MMG sensor 301. This sensor functions in a similar way to that shown in FIG. 2a but has a few structural differences. The case 311 of the previous sensor 301 is housed within a clip 317 for attachment to the patient's clothing (although other attachment members could be used instead), and the isolation chamber 313 is filled with silicone rather than glue. The MMG sensor 301 also has an integrated pressure sensor 308 for providing an indication of the contact pressure. In this example, the pressure sensor 308 is positioned between the case 311 and the clip 317 such that the contact pressure causes compression of the pressure sensor 308. With this configuration, the pressure sensor 308 provides an indication of compression of the mechanomyography sensor. The printed circuit board (PCB) 318 on which the microphone 314 is formed is also shown.

Rather than using a spatial modulation pressure bias system 203, the present apparatus 200 may incorporate a pressure bias system that is configured to temporally modulate the applied pressure of at least some of the MMG sensors 201 in the sensor array. In this scenario, the temporal modulation of applied pressure may be effected by at least some of the MMG sensors 201 having an adjustable height relative to a reference plane of the substrate 202 (e.g. the upper surface 210 of the substrate 202).

The temporal modulation may be applied globally across all MMG sensors 201 of the array simultaneously, or it may be applied locally with respect to one or more specific MMG sensors 201. A global application of temporal modulation may be achieved using a substrate 202 that is configured to be worn around the body (e.g. in a bracelet or armband form) and a pressure bias system that comprises means for adjusting the tightness of the substrate 202 around the body. In this scenario, the substrate 202 could be formed from a flexible or stretchable material, or it may comprise a plurality of rigid substrate portions linked together by flexible/stretchable connectors. Furthermore, the means for adjusting the tightness of the substrate 202 around the body could comprise one or more of a fastener and an inflatable chamber attached to the substrate (similar to a blood pressure cuff).

With a global application of the temporal modulation, the MMG measurements may be repeated at different levels of contact pressure for all MMG sensors 201. With a local application of the temporal modulation, on the other hand, a single measurement with all sensors 201 simultaneously may be sufficient if MMG signals can be detected at different contact pressures for the same muscle or muscle group. The latter would depend on the spatial proximity of the MMG sensors 201 on the substrate 202.

FIGS. 4a and 4b show one example of a local temporal modulation pressure bias system in first and second states, respectively. In this example, the pressure bias system comprises an inflatable element 418 configured for adjusting the height of an MMG sensor 401 (and its case 411) relative to the reference plane of the substrate. The inflatable element 418 expands from the first (deflated) state to the second (inflated) state and pushes the MMG sensor 401 (and its case 411) further out of the clip 417. This movement of the MMG sensor 401 applies a greater contact pressure on the body which is detected by the pressure sensor 408.

FIGS. 5a and 5b show another example of a local temporal modulation pressure bias system in first and second states, respectively. In this example, the inflatable element 418 is replaced by a linear actuator 519. Under manual or automatic control, the linear actuator 519 changes dimension from the first (retracted) state to the second (extended) state and pushes an MMG sensor 501 (and its case 511) further out of the clip 517. As with the previous example, this movement of the MMG sensor 501 applies a greater pressure on the body which is detected by the pressure sensor 508.

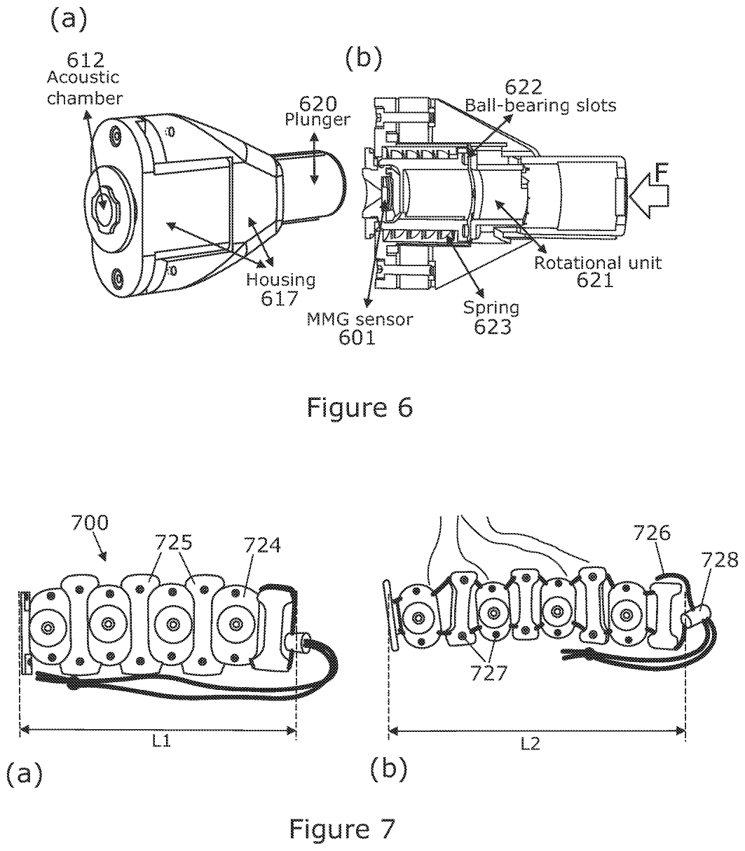

FIGS. 6a and 6b show yet another example of a local temporal modulation pressure bias system in perspective and cross-sectional views, respectively. As per the previous example, the pressure bias system incorporates a linear actuator which pushes an MMG sensor 601 further out of the clip/housing 617. This time, however, a rotational mechanism allows the contact pressure to be manually set to three different levels. When a force F is applied to the end of a plunger 620, the MMG sensor 601 increasingly protrudes from the base of the housing 617 in a linear stepwise manner. By turning a rotational unit 621 once the plunger 620 has reached a corresponding slot 622, the MMG sensor 601 can be set to three different positions each applying a corresponding level of contact pressure to the body. The use of ball bearings within the slots 622 facilitates rotation of the rotational unit 621 whilst preventing rotation of the MMG sensor 601 itself. A spring 623 enables the MMG sensor 601 to be retracted from the third (fully extended) position back to the first (fully retracted) position.

The present apparatus may be used to determine at least one of muscle activity, neural activity, neuronal activity and cerebral activity associated with one or more muscles. In this context, “muscle activity” includes one or more biomechanical properties of the muscles, e.g. the state of contraction and the action of going from one state of contraction to another. Furthermore, the neural, neuronal and cerebral activity includes the original neural signals which invoke the muscle activity. The neural signals may be determined using the present apparatus without necessarily having to determine the muscle signals.

In view of the above, the present apparatus has a variety of different applications. Examples include the evaluation of muscle fatigue, muscle strength, balance, muscle functions, and analysis of mechanical muscle responses during exercise. Other applications include the examination of neuromuscular disorders and prosthetic limb/robotic control. Regarding prosthetic control, experiments have shown that the present apparatus may be used to improve the performance and accuracy of gesture (or gesture intent) detection. These experiments will now be described.

FIGS. 7a and 7b show the present apparatus 700 used for the experiments. The apparatus 700 takes the form of a wearable armband comprising four MMG segments 724 spaced apart from one another by separation segments 725 on an elastic cord 726. The four separation segments 725 prevent the MMG segments 724 from shifting by maintaining the tension on the elastic cord 726. Each MMG segment 724 comprises an acoustic MMG sensor as described with reference to FIG. 3a and a temporal modulation pressure bias system as described with reference to FIGS. 6a and 6b. The acoustic MMG sensor has a conical shape, a diameter of 7 mm, a height of 5 mm and a 3 μm thick mylar film.

In each one of the segments 724, 725, two housing parts are coupled together by socket head cap screws 727 to form slots through which the elastic cord 726 is passed in order to hold the segments 724, 725 together. This design allows each MMG segment 724 to be slid along the cord 726 so that it can be placed over the target muscles. Depending on the arm diameter of each individual, the active length of the elastic cord 726 can be adjusted using a spring buckle 728 prior to the experiment to ensure the same tension values on each MMG segment 724 for every participant. The length of the armband can be adjusted to values ranging from 13.5 cm (L1) to 28.5 cm (L2). In this study, the length of the armband was tuned and normalized based on the diameter of the limb of the participant to maximize the similarity of conditions. It is assumed that slight differences in the initial conditions caused when adjusting the length of the armband to each subject's arm diameter did not significantly affect the quality of the MMG activity and can therefore be neglected.

Six abled-bodied right-handed individuals (4 males and two females) between the ages of 19 to 35 years old and three amputees (2 males and one female) between the ages of 30 to 50 years old participated in this experiment that took place on a single session per participant. The experiments involved the collection of MMG activity while participants were asked to perform different hand gestures. Able-bodied participants were asked to perform six different gestures, and amputees were asked to perform only four of them.

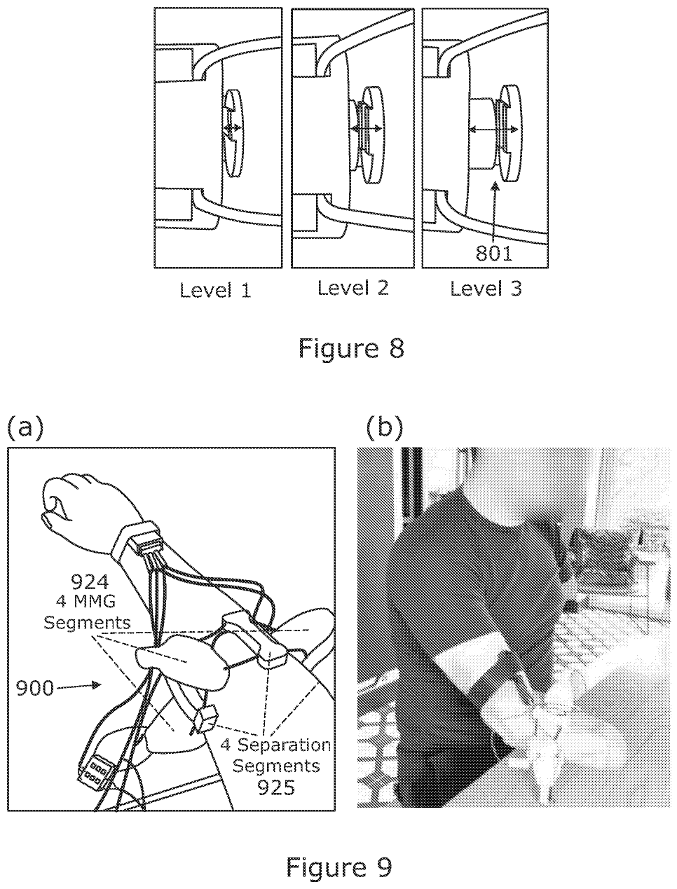

FIG. 8 shows the MMG sensor 801 at three different positions to provide three different levels of contact pressure. All three levels of contact pressure were tested on able-bodied participants whilst only the first two levels of contact pressure were tested on amputee participants.

FIGS. 9a and 9b show the stretchable armband as applied to an able-bodied participant and an amputee participant, respectively. The participants were asked to sit with their dominant arm placed over an armrest. The armband 900 was positioned around the forearm (i.e. the “body” for this study), and the MMG segments 924 were moved accordingly in order to be placed over the flexor carpi radialis, brachioradialis, extensor digitorum and flexor carpi ulnaris muscles, with the separation segments 925 positioned therebetween.

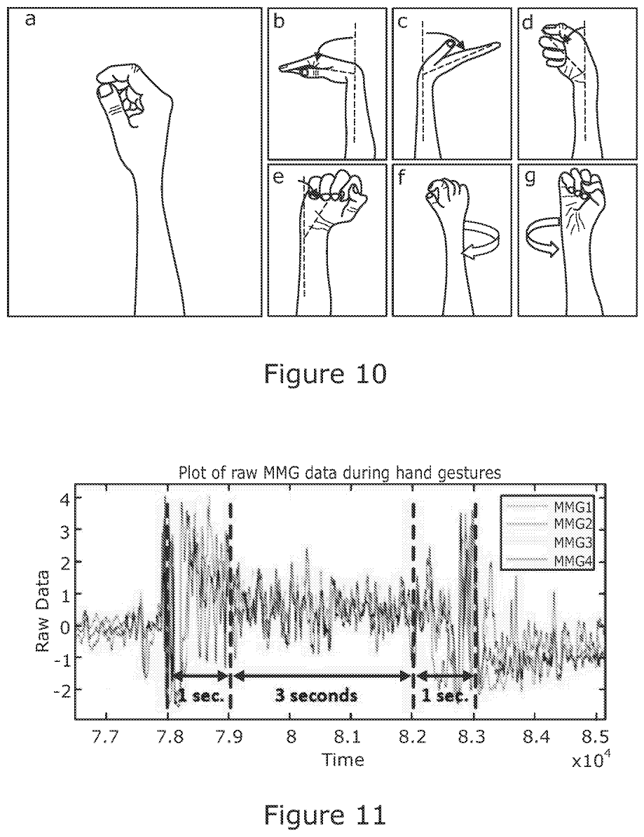

FIGS. 10a-g show the different hand gestures used during the experiment. As a starting position, the participant's dominant hand was positioned at rest mid-way in supination at the forearm with the hand approximately 5 cm above from the surface of the table (FIG. 10a). The seat was adjusted for the participant to rest their elbow at approximately 90°. During data collection, the six different hand gestures chosen for able-bodied participants were: Flexion (FIG. 10b), Extension (FIG. 10c), Adduction (FIG. 10d), Abduction (FIG. 10e), Pronation (FIG. 10f) and Supination (FIG. 10g). The four gestures chosen for amputees were Flexion, Extension, Pronation, Supination.

Data collection was divided into three groups with a 30-second resting period between each recording. Groups were divided as follows: Flexion-Extension, Pronation-Supination, and Adduction-Abduction. Only the first two groups applied to the amputees. Each group consisted of 5 repetitions of each gesture. Initially, participants were asked to place the hand in the resting position. After initiating data collection, participants were asked to maintain the resting position for a period of 10 seconds before carrying out the first contraction. Participants were then asked to perform the sustained contractions for a period of 5 seconds with a 5-second interval to rest between each contraction. At the end of each trial, participants were asked to maintain the hand at rest for a period of 10 seconds before stopping the recording in order to facilitate data extraction. After the three groups of recordings had finished, each of the MMG sensors were pushed out to the next level to increase the amount of contact force. The process of data recording for the three groups of hand gestures was then performed again. This process was repeated twice for able-bodied participants in order to record data for all hand gestures at three levels of contact pressure. In order to avoid the muscle fatigue, and due to time restrictions, the amputee data collection process was repeated only once in order to collect data for all gestures at two levels of contact pressure.

FIG. 11 shows the raw MMG signals detected during the hand gestures. The signals were divided into two sets for the purpose of training and testing a classifier. For this study, a hold-out validation method was chosen since it shows a more realistic view of the performance of the apparatus. In this regard, the testing was done on a completely unseen dataset to maximize the validity of the technique and strictly avoid any potential unwanted information leak between the test and training sets. The first set (for training) contained the first three trials of each hand gesture, and the second set (for testing) contained the remaining two trials.

The signal was analysed during the steady-state phase of the contraction only, thus discarding the transient phases which have a high degree of stochastic non-stationarity. This makes the steady state of the signal more robust for classification and gesture detection purposes. In addition, the duration of the transient phases cannot be controlled accurately for systematically training of the machine learning algorithms under practical situations without rigorous and excessive calibration.

For each trial, the onset and offset of each gesture were marked, and data for the first and last second was discarded. The information for the remaining 3 seconds in between was extracted for further analysis (see FIG. 11). Most of the information context of an MMG signal is below 100 Hz, and frequencies between 1 Hz and 25 Hz typically contain most of the signal power. As such, the region of interest for frequency analysis of the MMG signal falls between 1 Hz to 100 Hz. The signals were filtered using a second-order band-pass Butterworth filter and then passed through a Hilbert transform function. This data processing was performed for both the training and testing data sets.

In this work, a total of 213 spectrotemporal features were extracted for each channel, including 200 features in the frequency domain and 13 features in the time domain. Data was segmented into windows of 200 ms with no overlapping. The frequency features were extracted using fast Fourier transform. The time domain features included: Root Mean Square, Integrated Absolute Value, Mean Absolute Value, Modified Mean Absolute Value type 1, Modified Mean Absolute Value type 2, Simple Square Integral, Variance, The 3rd Temporal Moment, The 4th Temporal Moment, The 5th Temporal Moment, Average Amplitude Change, Difference Absolute Standard Deviation Value and Difference Absolute Mean.

After feature extraction, a Neighbourhood Component Analysis (NCA) was applied in order to extract the most relevant features for classification purposes. NCA is a relatively new feature scoring technique that assigns a power weight to each feature based on the discriminative power of the corresponding feature. This technique enables ranking of the features, comparing the importance of each feature based on the discriminative power and selecting features which contain most of the power for classification. After feature scoring and selection using a holdout validation method, features from the first data set were used to train a linear support vector machine (SVM) classifier, and the second data set was used to test the performance of the model.

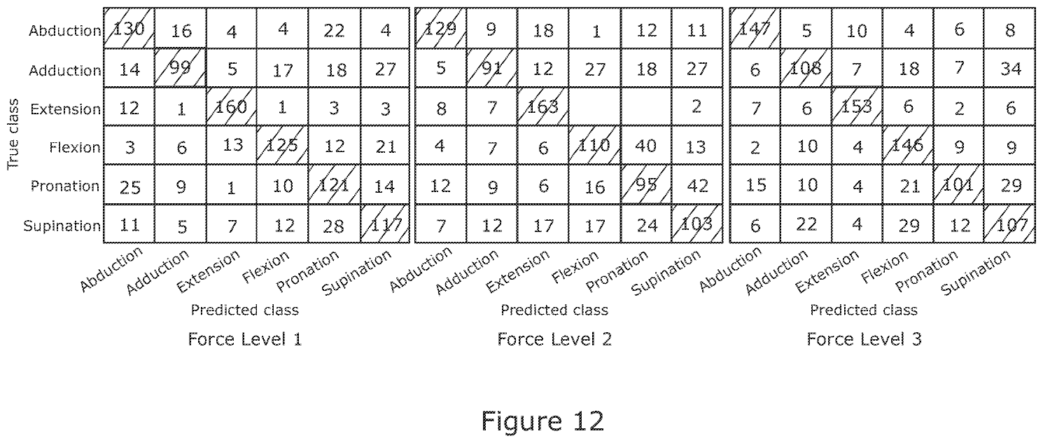

FIG. 12 shows the average confusion matrices for each level of contact pressure applied during the experiment. At the first level of contact pressure (force level), gesture classification was strongest for Extension and weakest for Adduction. Adduction, Abduction, and Flexion were best classified at the third level of contact pressure. In contrast, the classification accuracy of Extension decreased from the second level of contact pressure to the third level of contact pressure despite outperforming the other gestures at the third level.

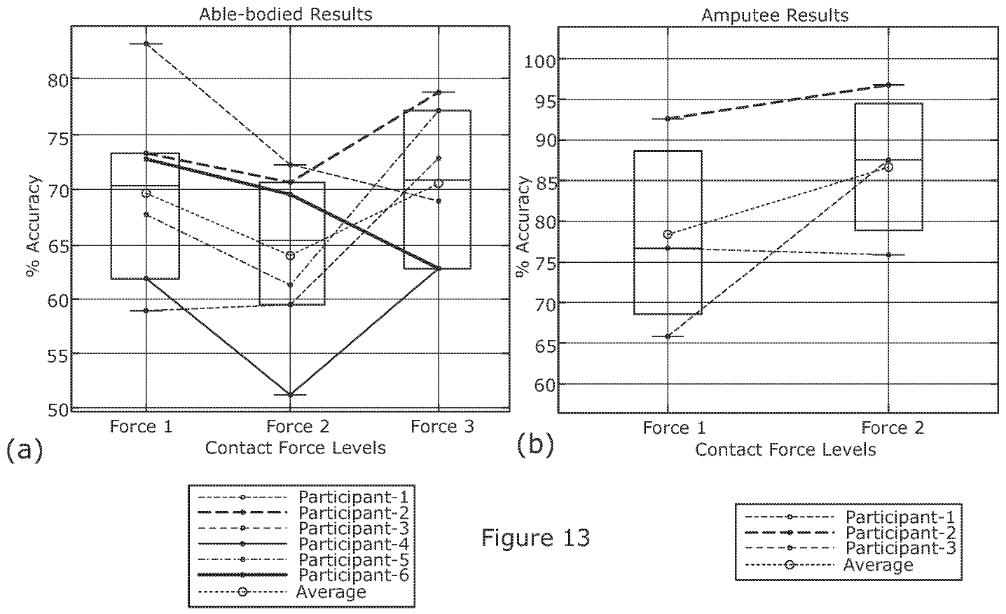

FIGS. 13a and 13b show the percentage accuracy of the classifier vs level of contact pressure for able-bodied and amputee participants, respectively. For amputee participants, the average accuracy of the classifier was 78.33% for contact pressure (force) level 1 and 86.67% for contact pressure (force) level 2. An average improvement in accuracy of 8.34% was observed with a standard deviation of 0.13 and 0.10 by increasing the contact pressure level from 1 to 2, respectively. The change in the pattern of improvement was user specific. For amputee participant #3, an increase of the contact pressure increased the accuracy by about 25%, whilst an improvement near to 6% was observed for amputee participant #2. For amputee participant #1, the performance remained almost the same.

For the able-bodied participants, increasing the contact pressure from the first level to the second level resulted in a 5.65% decrease in the average accuracy, but increasing the contact pressure to the third level caused an average increase of 6.58%. For Participant #2 of the able-bodied group, increasing the contact pressure from the first level to the second level reduced the performance from 73% to 70% but further increasing the contact pressure to the third level improved the accuracy from 70% to almost 80%.

A more intense clinical evaluation will be needed to assess the performance of the system on a large population of patients and disabled users. Nevertheless, the current results support a relationship between the level of contact pressure and the performance of MMG-based gesture detection. It suggests that the accuracy is not linearly increased by the contact pressure. Rather, each participant may have an optimal contact pressure for maximising the MMG performance. The results on two out of the three amputees tested also illustrate the benefits of the present apparatus. Due to the user-specific signature of performance and the corresponding nonlinear dependency on the contact pressure, an average behaviour may not be representative of the overall performance.

An analysis of the signal-to-noise ratio (SNR) was performed at each level of contact pressure for every participant (not shown). The average SNR values across all able-bodied participants was 5.95 dB, 5.87 dB, and 6.63 dB for contact pressure levels of 1, 2, and 3, respectively. In the case of the amputee data, the average SNR values were 5.76 dB and 5.86 dB for contact pressure levels 1 and 2, respectively. A calculation of the bandwidth was also performed for each level of contact pressure and averaged across all participants (not shown). The estimated occupied bandwidths of the signals from able-bodied participants for contact pressure levels 1, 2, and 3 were 24.33, 24.86, and 22.04, respectively. The estimated bandwidths of the signals from amputee participants for contact pressure levels 1 and 2 were 28.08 and 27.83, respectively. It should be highlighted, however, that the focus of this study was on the discriminative power of the signal space (in particular, for the data collected from amputee users). This may relate in an indirect manner to the SNR value and signal bandwidth. The results provide insight into the potential effect of different levels of contact force on the MMG signal, SNR and bandwidth. A gradual improvement of the SNR value and decrease in the bandwidth was observed, as stated above.

Although the above description relates to an array of MMG sensors, another example of the present apparatus (not illustrated) may comprise one or more MMG sensors configured to detect MMG signals from the body to which the apparatus is applied, and one or more pressure sensors configured to provide an indication of contact pressure of a respective MMG sensor on the body surface (with or without a temporal modulation pressure bias system). This combination not only enables quantitative tracking (and possibly tuning) of the contact pressure, but also allows measurement of force myography (FMG) signals for fusion with the MMG signals as a potential solution for detecting the transient phases of muscle contraction. In this example, the apparatus may also comprise processing circuitry configured to associate the detected MMG signals with the indicated contact pressure for use in determining activity associated with a muscle located within the body. Association of the detected MMG signals and indicated contact pressure may be based on timing information. The contact pressure throughout the relevant MMG signal is required, and thus, the signals from the MMG sensors should be synchronised with the signals from the pressure sensors. For example, when classifying using segmented data, the average contact pressure during detection of the segmented MMG signal can be used. Here the term “segmented” defines the part of the signal currently being examined by the system. So, for example, if a 200 ms segment of the MMG data is being considered, features from the same 200 ms segment of the pressure data should be included (and average pressure could be one of those features).

Additionally or alternatively, the processing circuitry may be configured for fusion of the MMG and FMG signals as mentioned above. Signal fusion may be used to maintain consistency between the training and test data sets. For example, both signals could be used as inputs to a deep learning algorithm such as a convolutional neural network. The contact pressure data could be used to select training data from the training set which was taken at a similar (relevant) contact pressure. Contact pressure could also be used to configure the cut-off frequency for the MMG sensors, since changing contact pressure can (in some circumstances) modify the frequency response of a muscle. Contact pressure may further be used to configure a frequency equaliser to amplify specific relevant frequencies, or to normalise/calibrate between data taken at different contact pressures where contact pressure has affected the frequency response of the muscle.

Yet another example of the present apparatus (not illustrated) may comprise an array of MMG sensors spatially distributed across a substrate with a spacing of no more than 20 mm (and in some cases, no more than 5 mm, 10 mm or 15 mm), each configured to detect MMG signals from the body to which the apparatus is applied. In this example, the apparatus may or may not comprise a pressure bias system for modulating the contact pressure, and may or may not comprise an array of pressure sensors for measuring the contact pressure of respective MMG sensors. Nevertheless, this arrangement does allow multiple MMG sensors to detect MMG signals from the same muscle or muscle group, thereby increasing the spatial resolution and robustness against defective sensors. The apparatus may also comprise processing circuitry (e.g. a decomposition module running an associated computer program) configured to decompose the MMG signals detected by the array of MMG sensors for use in determining neural activity associated with one or more muscles located within the body. In this scenario, the processing circuitry decomposes the signals from many data streams representing the combined muscle signals to individual data streams each representing a signal from a single muscle. Furthermore, since each motor unit is controlled by a single nerve, it is possible to determine information about the neural activity triggering a motor unit contraction from signals obtained from a single motor unit. Such decomposition requires a relatively high spatial density of MMG sensors (e.g. a spacing of no more than 20 mm) to be able to detect the same contraction, as the subtle differences in position introduce distortions in the way the signals combine.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/embodiments may consist of any such individual feature or combination of features. In view of the foregoing description it will be evident to a person skilled in the art that various modifications may be made within the scope of the disclosure.

1. An apparatus configured for application to a surface of a body, the apparatus comprising:
an array of mechanomyography sensors spatially distributed across a substrate, each mechanomyography sensor configured to detect mechanomyography signals from the body to which the apparatus is applied; and
a pressure bias system configured to provide a variation in contact pressure of the mechanomyography sensors to the body surface to receive mechanomyography signals at different levels of applied contact pressure.

2. The apparatus of claim 1, wherein the pressure bias system is configured to: (a) spatially modulate the applied pressure across the sensor array; or (b) temporally modulate the applied pressure of at least some of the mechanomyography sensors in the sensor array.

3. The apparatus of claim 2, wherein the spatial modulation of applied pressure is effected by a plurality of the mechanomyography sensors in the sensor array being distributed at different distances from and orthogonal to a reference plane of the substrate.

4. The apparatus of claim 3, wherein the pressure bias system comprises a plurality of platforms on the substrate at different heights relative to the reference plane of the substrate, each platform bearing at least one mechanomyography sensor of the sensor array.

5. (canceled)

6. The apparatus of claim 2, wherein the temporal modulation of applied pressure is effected by at least some of the mechanomyography sensors having an adjustable height relative to a reference plane of the substrate.

7. The apparatus of claim 6, wherein the pressure bias system comprises one or more actuators or inflatable elements configured for adjusting the height of at least one mechanomyography sensor relative to the reference plane of the substrate.

8. The apparatus of claim 2, wherein the substrate is configured to be worn around the body, and wherein the pressure bias system comprises means for adjusting the tightness of the substrate around the body and optionally, wherein the substrate comprises a plurality of rigid substrate portions linked together by flexible and/or stretchable connectors.

9. The apparatus of claim 8, wherein the means for adjusting the tightness of the substrate around the body comprise one or more of a fastener and an inflatable chamber attached to the substrate.

10. The apparatus of claim 9, wherein the substrate comprises a plurality of rigid substrate portions linked together by flexible and/or stretchable connectors.

11. The apparatus of claim 11, further comprising:
(a) an array of pressure sensors, each pressure sensor configured to provide an indication of the contact pressure of a respective mechanomyography sensor as applied to the body surface;
and/or (b) an array of electromyography electrodes disposed on the substrate, each electromyography electrode configured to detect electromyography signals from the body to which the apparatus is applied.

12. (canceled)

13. The apparatus of claim 1, wherein: (a) the array is a two-dimensional array; (b) the array is a regular array; and/or (c) the sensors of the array have a spacing of no more than 5 mm, 10 mm, 15 mm or 20 mm.

14. (canceled)

15. (canceled)

16. The apparatus of claim 1, wherein the apparatus comprises processing circuitry configured to process the mechanomyography signals detected from the body at different levels of applied contact pressure to determine activity associated with one or more muscles located within the body.

17. The apparatus of claim 16, wherein the determined activity associated with the one or more muscles within the body comprises at least one of muscle activity, neural activity, neuronal activity and cerebral activity.

18. The apparatus of claim 17, wherein the determined muscle activity comprises one or more biomechanical properties of the muscles.

19. The apparatus of any of claim 16, wherein: (a) the processing circuitry is configured to process the detected mechanomyography signals to determine activity associated with one or more muscles located at different corresponding depths within the body; and/or (b) the processing circuitry is configured to process the detected mechanomyography signals to detect the frequency response of one or more muscles at different levels of applied contact pressure.

20. (canceled)

21. The apparatus of claim 16, further comprising an array of pressure sensors, each pressure sensor configured to provide an indication of the contact pressure of a respective mechanomyography sensor as applied to the body surface, wherein the processing circuitry is configured to associate mechanomyography signals received from the mechanomyography sensors with pressure signals received from the respective pressure sensors.

22. The apparatus of claim 21, wherein: (a) the processing circuitry is configured to associate the mechanomyography signals with the pressure signals by synchronising read-out of the mechanomyography sensors with read-out of the respective pressure sensors; and/or (b) the processing circuitry is configured to fuse the mechanomyography signals with the pressure signals.

23. (canceled)

24. An apparatus configured for application to a surface of a body, the apparatus comprising:
a mechanomyography sensor configured to detect mechanomyography signals from the body to which the apparatus is applied;
a pressure sensor integrated with the mechanomyography sensor and configured to provide an indication of contact pressure of the mechanomyography sensor on the body surface; and
processing circuitry configured to calibrate the detected mechanomyography signals using the indicated contact pressure for use in determining activity associated with a muscle located within the body.

25. The apparatus of claim 24, wherein the apparatus comprises a casing configured to house the mechanomyography sensor, and an attachment member for attaching the casing to the body, and wherein the pressure sensor is positioned between the casing and the attachment member such that the contact pressure causes compression of the pressure sensor.

26. An apparatus configured for application to a surface of a body, the apparatus comprising:
an array of mechanomyography sensors spatially distributed across a substrate with a spacing of no more than 20 mm, each mechanomyography sensor configured to detect mechanomyography signals from the body to which the apparatus is applied; and
processing circuitry configured to decompose the mechanomyography signals detected by the array of mechanomyography sensors for use in determining neural activity associated with one or more muscles located within the body.
